(19) United States
(12) Patent Application Publication
(10) Pub. No.: US 2013/0250236 A1
(43) Pub. Date: Sep. 26, 2013

(54) GAZE-FIXATION AIDING AND IMAGE FOCUSING DEVICE

(71) Applicant: CRYSTALVUE MEDICAL CORPORATION, Taoyuan County (TW)

(72) Inventors: Che Liang TSAI, Taichung City (TW); Chung Ping CHUANG, Taoyuan City (TW); Yen Jen CHANG, Kaohsiung City (TW)

(73) Assignee: CRYSTALVUE MEDICAL CORPORATION, TAOYUAN COUNTY (TW)

(21) Appl. No.: 13/722,616

(22) Filed: Dec. 20, 2012

(30) Foreign Application Priority Data
Mar. 23, 2012 (TW) 101110016

(51) Int. Cl. A61B 3/14

(52) U.S. Cl. CPC A61B 3/14
USPC 351/206

(57) ABSTRACT

A gaze-fixation aiding and image focusing device for a fundus camera includes an illuminating system for projecting an examination light to illuminate an examinee's fundus; an aging system for receiving a fundus image and light reflected from the examinee's eye and forming images of the reflected light and the fundus on a display; a focusing device having a split image screen located in the illuminating system to work with an adjusting means located in the imaging system for split image focusing; and a gaze fixation device having a gaze fixation surface formed in the illuminating system and a plurality of fixation points provided on the gaze fixation surface to form a contrast with the examination light. With these arrangements, the fundus camera can have largely simplified optical path structure, and the fixation points are independently controllable to light for the examinee to gaze into particular directions.

### FIELD OF THE INVENTION

The present invention relates to a gaze-fixation aiding and image focusing device employed in fundus examination, and more particularly to a fundus camera having a gaze fixation device and a focusing device integrated into an illuminating system and an imaging system, respectively.

### BACKGROUND OF THE INVENTION

A fundus camera is a camera particularly used for capturing images of inner eye and retina. Compared to other objects in photographing, the fundus does not give light itself. Light of different types projected onto an eye in a natural condition can illuminate the fundus but is not intense enough for observing the fundus or taking a picture thereof. Besides, light tends to reflect from the cornea to interfere with the observation of the fundus.

Therefore, the fundus camera must include an illuminating system capable of illuminating the fundus with a considerably intense light as well as an observing and imaging system that is free from any influence of the intense reflected light from the cornea on a film. Further, to distinguish the position of the macular or the optic disc from other areas in the captured eye image, fixation points are provided in the fundus camera so that an examinee can turn his or her eye to a particular angle by gazing on some specific fixation point.

Please refer FIG. 1. The gaze fixation system adopted by the conventional fundus camera mainly includes a liquid crystal display (LCD) 101, on which lightened spots 102 are provided at different positions. In using the LCD gaze fixation system, an examinee is caused to turn his or her eye and gaze on the lightened spots 102 on the LCD 101. Please refer to FIG. 2. The fundus camera using the conventional LCD gaze fixation system has an optical path design generally divided into an LCD display system 10, a light source projecting system 11, an optical camera system 12, an image displaying and monitoring system 13, and a positioning optical path system 14.

The LCD display system 10 includes an LCD display 101, a condenser lens 103 and a beam splitter 104. The light source projecting system 11 includes a photographing light source 111, a condenser lens 112, a ring-shaped slit plate 113, a relay lens 114 and a perforated mirror 115. The optical camera system 12 includes an ocular lens 121, a focusing lens 122, a magnifier 123 and a film 124. The image displaying and monitoring system 13 includes a beam-bending lens 131, a field lens 132, a mirror 133, a relay lens 134, a converter tube 135 and a monitor 136. The positioning optical path system 14 includes a semi-lens 141, a relay lens 142, a mirror 143, a light guide 144 and a light source 145.

Since the above optical path systems are independent from one another, they respectively require an independent optical path space. As a result, the fundus camera requires increased manufacturing cost. Further, the LCD for aiding gaze fixation does not include any optical path design related to a split image focusing screen. Therefore, an examiner could not quickly and precisely focus the image in the process of image focusing adjustment for individual eye diopter. However, the addition of a split image focusing system to the LCD display system would inevitably involve in the complicated problem of coordination with other optical path systems.

In view that there are still many improvements that must be made to the conventional fundus camera adopting the LCD for aiding gaze fixation, it is tried by the inventor to develop an improved gaze-fixation aiding and image focusing device for the fundus camera.

### SUMMARY OF THE INVENTION

A primary object of the present invention is to provide a gaze-fixation aiding and image focusing device for helping an examinee in eye rotation and gaze fixation to thereby enable convenient image focusing by an examiner, which integrates a gaze fixation device and a focusing device into an illuminating system and an imaging system, respectively, to enable effectively simplified optical path design of a fundus camera.

Another object of the present invention is to provide a gaze-fixation aiding and image focusing device that allows an examiner to make split image focusing adjustment according to individual eye diopter, and allows different gaze fixation targets to be independently controlled to light or extinguish according to different requirements in an eye examination, so that an examinee can rotate his or her eye to a particular angle in response to a showed gaze fixation target.

A further object of the present invention is to provide a gaze-fixation aiding and image focusing device that includes a gaze fixation device using invisible light as an examination light and intermittently lightened spots as fixation points to build an examination condition having a high contrast between a black background and the intermittently lightened spots, so that an examinee's eye is not stimulated by light and can more easily and comfortably gaze on the fixation points.

A still further object of the present invention is to provide a gaze-fixation aiding and image focusing device that has fixation s and a split image screen integrated onto one gaze fixation surface, which can be configured to have a curvature the same as that of a human eye, so that the fixation points are located at clearly recognizable focused position when a split image focusing is completed, allowing an examinee to gaze on the fixation points during an eye examination.

To achieve the above and other objects, the gaze-fixation aiding and image focusing device according to the present invention includes an illuminating system, an imaging system, a focusing device and a gaze fixation device. The illuminating system has an illuminating optical path, along which an examination light is projected to illuminate an examinee's fundus. The imaging system has an imaging optical path for receiving the examinee's fundus image and light reflected from the examinee's eye, and for forming images of the reflected light and the examinee's fundus on a display. The focusing device includes a split image screen located in the illuminating optical path and an adjusting means located in the imaging optical path to work with the split image screen to achieve split image focusing. The adjusting means is configured as a movable focusing lens for selectively adjusting a focal length position. The split image screen includes a light blocking plate of a predetermined size having a light-transmissible slit provided thereon, and two facing prisms arranged in front of the slit. The gaze fixation device includes a gaze fixation surface formed in the illuminating optical path for the examinee to gaze on and a plurality of fixation points provided on the gaze fixation surface with a contrast being created between the examination light and the fixation points.

The split image screen and the fixation points are both located on the gaze fixation surface to together form a light-impenetrable zone that blocks the illuminating optical path, and areas on the gaze fixation surface other than the light-impenetrable zone together form a light-penetrable zone. The examination light projected along the illuminating optical path passes through the light-penetrable zone into the examinee's eye.

According to the present invention, the split image screen and the movable focusing lens of the focusing device are integrated into the illuminating optical path and the imaging optical path, respectively; and the fixation points of the gaze fixation device are integrated into the illuminating optical path to locate on the same gaze fixation surface as the split image screen. In this manner, it is able to overcome the drawback in the conventional fundus camera, of which different functional systems respectively require an independent optical path space, so that the fundus camera can have largely reduced volume and lowered manufacturing cost.

According to an embodiment of the present invention, the examination light is an invisible light and the fixation points are respectively provided with at least one light-emitting unit that can be selectively controlled to light or extinguish. Further, the fixation points are connected to one another via a linking unit. The light-emitting units are electrically connected to the linking unit, so that control signals and electric power supply can be provided to the light-emitting units via the linking unit. Further, the light-emitting units can be independently controlled by the examiner to light or extinguish, so that an examination condition having a high contrast between a black background and the lightened spots can be built that allows an examinee to more easily and comfortably gaze on the fixation points.

In the present invention, the light-emitting units can be configured according to different examination requirements as light-emitting diodes, lamp tubes, light bulbs or optical fibers. Further, the light-emitting units can be in the form of geometrical shapes, characters, figures, letters, symbols, patterns or any combination thereof.

In an operable embodiment, the linking unit is a cross-shaped plate extended from the light blocking plate to each of the fixation points. In another operable embodiment, the linking unit includes a horizontal plate, which is integrally outward extended from two opposite lateral sides of the light blocking plate to link with the fixation points; a ring-shaped plate, which is integrally outward extended from two opposite ends of the horizontal plate; and two vertical plates, which are respectively radially inward extended from an upper and a lower end of the ring-shaped plate to link with the fixation points. In a further operable embodiment, the linking unit includes a vertical plate, which is integrally radially outward extended from a lower side of the light blocking plate to link with the fixation points; a ring-shaped plate, which is integrally extended from a lower end of the vertical plate to form a circle around the light blocking plate; and three projected plates integrally radially inward extended from an upper, a left and a right end of the ring-shaped plate to link with the fixation points.

In the present invention, the gaze fixation surface can be a flat vertical surface or a concave surface having a curvature corresponding to that of a human eye. In the case of the flat vertical gaze fixation surface, the fixation points are arranged on an upper, a lower, a left and a right side of the vertical gaze fixation surface to locate around and space from the split image screen by a predetermined distance and at two opposite lateral sides of the slit on the light blocking plate of the split image screen.

On the other hand, in the case of the concave gaze fixation surface, the fixation points are arranged on an upper front, a lower front, a front left and a front right side of the concave surface to locate around the split image screen and at two opposite lateral sides of the slit of the split image screen. When the gaze fixation surface is configured to have a curvature the same as that of a human eye, the fixation points can locate at clearly recognizable focused position after the split image focusing is completed.

According to an embodiment of the present invention, a contrast can be created between the examination light, which is an invisible light, and the light-emitting units. According to another preferred embodiment, the examination light can be a visible light and the fixation points are respectively provided with at least one lightproof unit. In this manner, it is also possible to effectively simplify the overall optical path system design of the fundus camera and help the examinee to turn his or her eye to particular angles in response to showed gaze fixation targets.

In an operable embodiment of the present invention, the light-penetrable zone is configured as a transparent substrate, the split image screen is arranged at a center of the transparent substrate, and the lightproof units are arranged on the transparent substrate to locate around and space from the split image screen by a predetermined distance.

### BRIEF DESCRIPTION OF THE DRAWINGS

The structure and the technical means adopted by the present invention to achieve the above and other objects can be best understood by referring to the following detailed description of the preferred embodiments and the accompanying drawings, wherein

FIG. 1 is a perspective view of an LED display with fixation points conventionally employed in fundus examination;

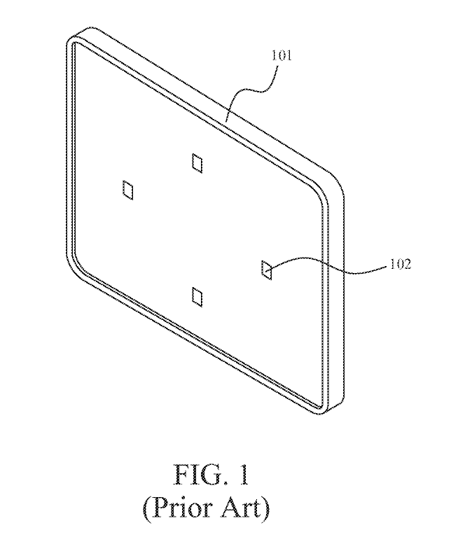

FIG. 2 is an optical path system diagram for a conventional fundus examination system employing the LED display of FIG. 1;

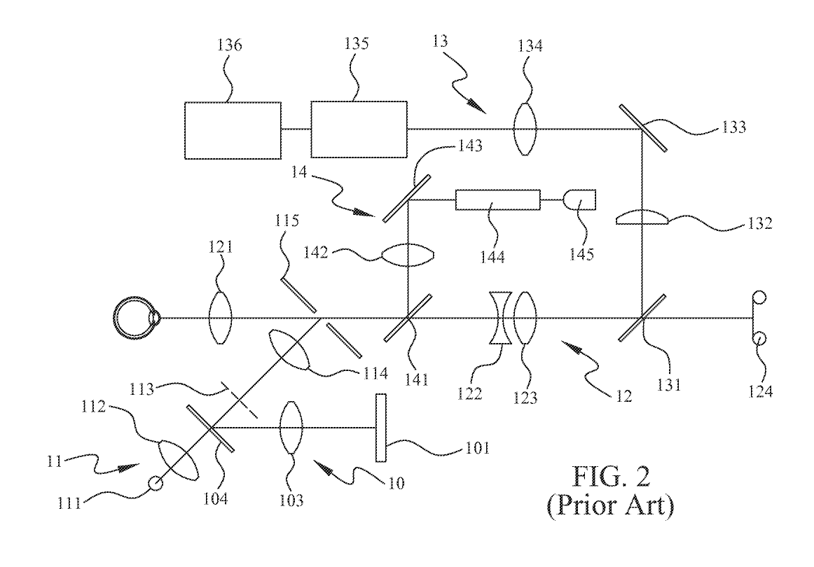

FIG. 3 is an optical path system diagram for a gaze-fixation aiding and image focusing device according to the present invention used in fundus examination;

FIG. 4 is a perspective view of an aiding element for the gaze-fixation aiding and image focusing device according to a first preferred embodiment of the present invention, in which a visible light is used as light source for eye examination;

FIG. 5 is a front view of an aiding element for the gaze-fixation aiding and image focusing device according to a second preferred embodiment of the present invention, in which an invisible light is used as a light source for eye examination;

FIG. 6 is a front view of an aiding element for the gaze-fixation aiding and image focusing device according to a third preferred embodiment of the present invention, in which an invisible light is used as a light source for eye examination;

FIG. 7 is a front view of an aiding element for the gaze-fixation aiding and image focusing device according to a fourth preferred embodiment of the present invention, in which an invisible light is used as a light source for eye examination; and

FIG. 8 is a perspective view of an aiding element for the gaze-fixation aiding and image focusing device according to a fifth preferred embodiment of the present invention, wherein the aiding element includes a concave gaze fixation surface.

### DETAILED DESCRIPTION OF THE PREFERRED EMBODIMENTS

The present invention will now be described with some preferred embodiments thereof and with reference to the accompanying drawings. For the purpose of easy to understand, elements that are the same in the preferred embodiments are denoted by the same reference numerals.

To facilitate clear description of the technical features of the present invention, terms appeared in the specification are first defined as below. Unless otherwise specified, the term “system” mentioned herein means an assembly of lens, prisms, mirrors, lightproof plates and other various units that are needed to transmit light between an examinee's eye 20, a light emitter 31 and a display 43 to form an image; and the term “optical path” mentioned herein means a path in any system along which light is transmitted from a light projection start point, such as the light emitter 31 and the examinee's eye 20, to a light projection end point, such as the examinee's eye 20 and the display 43.

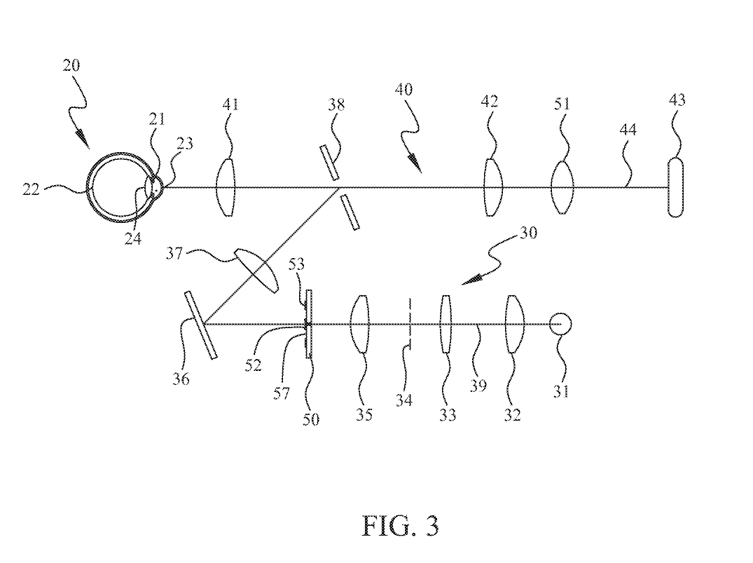

Please refer to FIG. 3. The present invention is a gaze-fixation aiding and image focusing device employed in a fundus camera mainly for capturing the image of an examinee's fundus 22 via the examinee's pupil 21 of eye 20. In an operable embodiment of the present invention, the gaze-fixation aiding and image focusing device includes a light emitter 31, a condenser lens 32, a first lens 33, a ring-shaped slit plate 34, a second lens 35, an aiding element 50, a mirror 36, a third lens 37, a perforated mirror 38, an ocular lens 41, a relay lens 42, a focusing lens 51, and a display 43 based on CMOS (complementary metal-oxide-semiconductor) sensor technology.

The light emitter 31, the condenser lens 32, the first lens 33, the ring-shaped slit plate 34, the second lens 35, the mirror 36, the third lens 37 and the perforated mirror 38 together constitute an illuminating system 30. On the other hand, the ocular lens 41, the relay lens 42 and the display 43 together constitute an imaging system 40.

In the illuminating system 30, the light emitter 31 projects an examination light source onto the condenser lens 32 and the first lens 33 to thereby form an image on the ring-shaped slit plate 34. The examination light then passes the ring-shaped slit plate 34 and is projected onto the mirror 36 via the second lens 35. The mirror 36 redirects the examination light to the third lens 37, via which the examination light is projected onto the perforated mirror 38. Finally, the perforated mirror 38 reflects the examination light onto an examinee's eye 20 to illuminate the examinee's fundus 22. Therefore, an illuminating optical path 39 from the light emitter 31 to the fundus 22 is formed in the illuminating system 30. In the above design, since the light emitter 31., the ring-shaped slit plate 34 and the examinee's pupil 21 of eye 20 are conjugate planes, allowing an examiner to precisely control an aperture size of the fundus camera for forming an image on the examinee's pupil 21 without being affected by size difference or a position deviation of the light emitter 31.

In the imaging system 40, the ocular lens 41 receives the examinee's fundus image and light reflected from the examinee's cornea 23 and eye-lens 24. The fundus image and the reflected light pass through a perforation on the perforated mirror 38 and the relay lens 42 to finally show on the display 43. Therefore, an imaging optical path 44 from the examinee's eye 20 to the display 43 is formed in the imaging system 40.

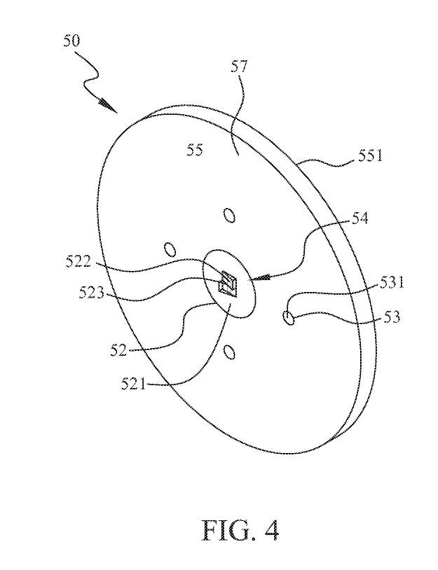

The aiding element 50 is located between the mirror 36 and the second lens 35 in the illuminating optical path 39. FIG. 4 shows the aiding element 50 in a first preferred embodiment of the present invention includes a split image screen 52 and a plurality of fixation points 53. The split image screen 52 cooperates with the focusing lens 51 to form a split image focusing device. The fixation points 53 and a contrast between them and the examination light together form a gaze fixation device for the examinee to turn the eye 20 to a particular angle in response to a gaze fixation target showed on the aiding element 50.

The split image screen 52 includes a light blocking plate 521 of a predetermined size, on which a light-transmissible slit 522 is provided; and two facing prisms 523 are arranged in front of the slit 522. The focusing lens 51 is located between the relay lens 42 and the display 43 to form an adjusting means for changing focal position. The examination light passes ⁻through the slit 522 and is changed in direction by the prisms 523 to produce two rectangular image parts on the display 43 for indicating whether an image is in focus.

In the first preferred embodiment of the present invention, the split image screen 52 and the fixation points 53 are located on the same plane to together form a light-impenetrable zone 54 that blocks the illuminating system 30. Areas of the aiding element 50 other than the light-impenetrable zone 54 together form a light-penetrable zone 55. The light-impenetrable zone 54 is made of an opaque material that is impenetrable by the examination light; and the light-penetrable zone 55 is made of a transparent material that allows the examination light to project into the examinee's eye. In the first preferred embodiment of the present invention, the light emitter 31 includes a visible light source and the above-mentioned examination light is a visible light; the light-penetrable zone 55 is configured as a transparent substrate 551; the opaque split image screen 52 is arranged at a center of the transparent substrate 551; and four lightproof units 531 are arranged around and spaced from the split image screen 52 by a predetermined distance to form four light-impenetrable fixation points 53. More specifically, the four fixation points 53 are spaced from the split image screen 52 to locate on an upper, a lower, a left and a right side thereof, respectively.

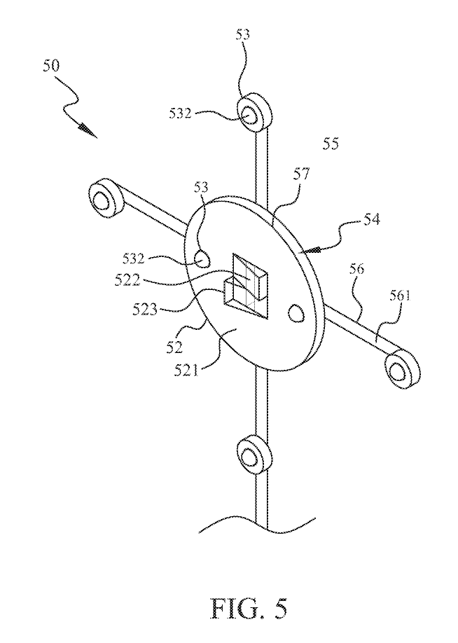

Please refer to FIG. 5 that shows the aiding element 50 in a second preferred embodiment of the present invention. In a final step of capturing the examinee's fundus image, a visible light is used. In view that the visible light would bring the examinee's pupil 21 to constrict, the present invention in the first preferred embodiment thereof uses the visible light and the lightproof units 531 to create a contrast between them. However, the present invention in the second preferred embodiment thereof can also use an invisible light in an alignment procedure that precedes the capturing of the fundus image, and the above-mentioned gaze fixation device and focusing device all are used in the alignment procedure, so that the examinee can see more clearly see the intermittently lightened fixation points on the invisible light background.

In the second preferred embodiment, the light emitter 31 includes a visible light source and an invisible light source, and the examination light comes from the invisible light source, such as an infrared light source. Further, the fixation points 53 are respectively provided with at least one light-emitting unit 532 that can be selectively controlled to light or extinguish. The light-emitting unit 532 can be configured as a light-emitting diode (LED).

In the second preferred embodiment, four fixation points 53 are arranged around and spaced from the split image screen 52 by a predetermined distance to locate on an upper, a lower, a left and a right side thereof, respectively, while two additional fixation points 53 are located at two opposite lateral sides of the slit 522 on the light blocking plate 521 of the split image screen 52. Further, a linking unit 56 is provided to connect the fixation points 53 to one another. The linking unit 56 is a cross-shaped plate 561 extended from the light blocking plate 521 to each of the fixation points 53. Open areas between the cross-shaped plate 561, the fixation points 53 and the light blocking plate 521 together form the above-mentioned light-penetrable zone 55.

The light-emitting units 532 are electrically connected to the linking unit 56, via which control signals and electric power supply are provided to the light-emitting units 532. In this manner, each of the fixation points 53 can be independently controlled by the examiner to light or extinguish. Further, the high contrast between the black background and the lightened fixation points allows the examinee to more easily and more comfortably gaze on the fixation points 53 during an eye examination.

When using the second preferred embodiment of the present invention to capture the fundus image, a photographer first projects an invisible light that is not perceivable with bare eyes. A small part of the invisible light passes through the split image screen 52 to form two rectangular image parts on the display 43 for indicating an image focusing condition, allowing the examiner to precisely focus on the examinee's fundus 22 without bringing the examinee's pupil 21 to constrict as a reflex response. On the other hand, the other large part of the invisible light passes through the light-penetrable zone 55 to irradiate on the fundus 22, enabling the photographer to make a preliminary observation of the examinee's eye 20. Since the invisible light does not stimulate photoreceptor cells in the eye, the examinee's pupil 21 would not constrict during the eye examination and an improved examination image can be obtained.

In case that the examiner needs to capture the image of the examinee's eve from a particular angle, the light--emitting units 532 at some specific fixation points 53 can be turned on, so that the examinee is guided to gaze on the specific fixation points 53 and accordingly adjusts the direction of gaze. The light-emitting units 532 can be differently configured to light continuously or intermittently, so as to give the examinee a clear target to gaze. With the second preferred embodiment of the present invention, there is only a black color within the examinee's field of view at first, but when the examiner turns on the light-emitting units 532 on the fixation points 53, the examinee is able to quickly and accurately aim at the lightened fixation points 53 in the black background.

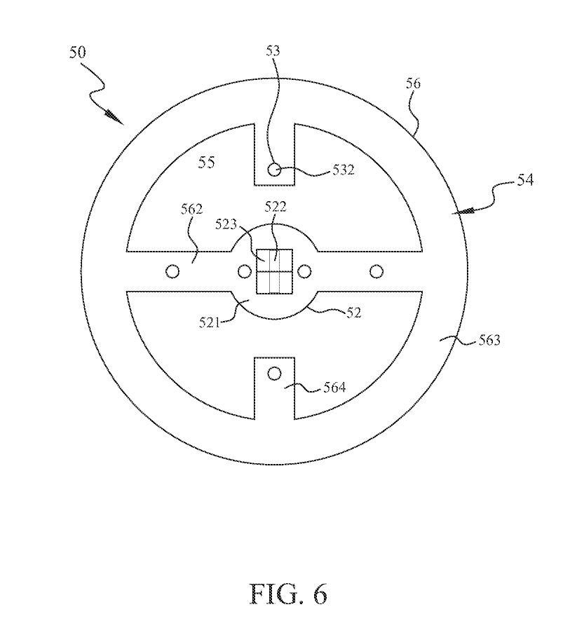

FIG. 6 shows the aiding element 50 in a third preferred embodiment of the present invention. In the third preferred embodiment, the examination light used and the positions of the fixation points 53 on the aiding element 50 are the same as those in the second preferred embodiment and are therefore not described in details herein. However, in the third preferred embodiment, the linking unit 56 for the aiding element 50 includes a horizontal plate 562, which is integrally outward extended from two opposite lateral sides of the light blocking plate 521 to link with the fixation points 53; a ring-shaped plate 563, which is integrally outward extended from two opposite ends of the horizontal plate 562; and two vertical plates 564, which are respectively radially inward extended from an upper and a lower end of the ring-shaped plate 563 to link with the fixation points 53. Areas of the aiding element 50 other than the horizontal plate 562, the ring-shaped plate 563, the vertical plates 564 and the light blocking plate 521 together form the above-mentioned light-penetrable zone 55.

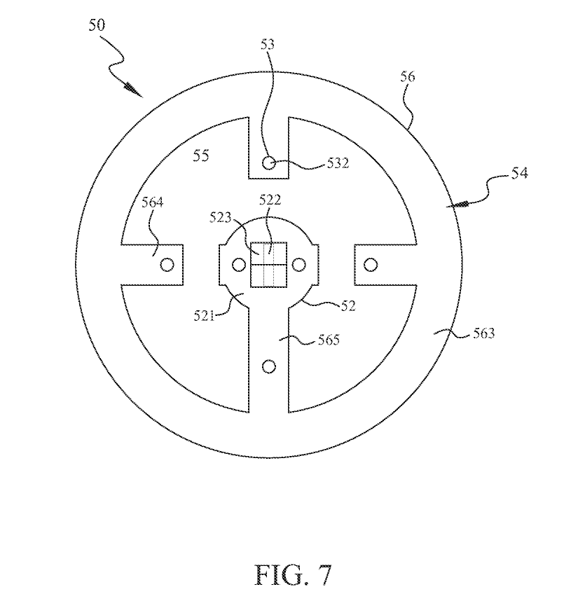

FIG. 7 shows the aiding element 50 in a fourth preferred embodiment of the present invention. In the fourth preferred embodiment, the examination light used and the positions of the fixation points 53 on the aiding element 50 are the same as those in the second and third preferred embodiments and are therefore not described in details herein. However, in the fourth preferred embodiment, the linking unit 56 for the aiding element 50 includes a vertical plate 565, which is integrally radially outward extended from a lower side of the light blocking plate 521 to link with the fixation points 53; a ring-shaped plate 563, which is integrally extended from a lower end of the vertical plate 565 to form a circle around the light blocking plate 521; and three projected plates 564 integrally radially inward extended from an upper, a left and a right end of the ring-shaped plate 563 to link with the fixation point 53. Areas of the aiding element 50 other than the vertical plate 565, the ring-shaped plate 563, the projected plates 564 and the light blocking plate 521 together form the above-mentioned light-penetrable zone 55.

The positions of the fixation points 53 and the type of the light-emitting units 532 described in the second, third and fourth preferred embodiments are only illustrative and not intended to limit the design of the light-emitting units 532. That is, the light-emitting units 532 can be otherwise configured as lamp tubes, light bulbs or optical fibers according to examination requirements. Further, in addition to the round shape as shown in the illustrated drawings, the light emitting units 532 may also be in the form of geometrical shapes, characters, figures, letters, symbols, patterns or any combination thereof.

As can be seen from FIGS. 4 to 7, the aiding element 50 in the first to the fourth preferred embodiment defines a flat vertical plane 57, on which the examinee gazes. However,since the human eye is actually a curved structure,it is possible what shown on the flat vertical plane 57 could not be clearly seen by the examinee.

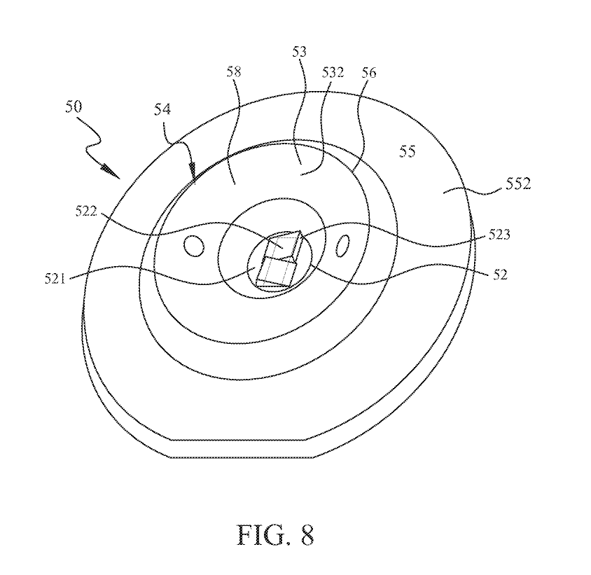

To overcome this problem, the aiding element 50 in a fifth preferred embodiment of the present invention, as shown in FIG. 8, includes a transparent curved substrate 552 corresponding to the curvature of a human eye. The transparent curved substrate 552 has a concave surface 58 formed thereon to serve as the above-mentioned linking unit 56. The fixation points 53 are arranged in an upper front, a lower front, a front left and a front right area of the concave surface 58 to locate around the split image screen 52; and the fixation points 53 are respectively configured as a light-emitting unit 532. The concave surface 58 and the split image screen 52 together constitute the above-mentioned light-impenetrable zone 54; and areas of the transparent curved substrate 552 other than the light-impenetrable zone 54 are made of a transparent material to form the above-mentioned light-penetrable zone 55. With these arrangements, the curved surface 58 is corresponding to the human eye curvature, and the fixation points 53 are located at an accurate fixation focal length that is obtained after image focusing via the split image screen 52 to enable increased clearness of the gaze fixation targets.

In summary, the present invention integrates the focusing device and the gaze fixation device into the illuminating system and the imaging system, respectively, to eliminate the drawback in the conventional fundus camera that different functional systems respectively require an independent optical path space. Therefore, the fundus camera with the present invention can have largely reduced volume to enable lowered manufacturing cost thereof. Moreover, in the present invention, the gaze fixation targets can be independently controlled to light or extinguish according to different requirements in eye examination, so that the examinee can turn his or her eye to a particular angle in response to each showed gaze fixation target and the examiner can conveniently capture the examinee's fundus images from different angles. In addition, the present invention uses invisible light as the examination light and uses intermittently lightened spots as the fixation points, so that the high contrast between the black background and the flashing spots enables the examinee to more easily and comfortably gaze on the fixation points. Further, in a preferred embodiment of the present invention, since the fixation points and the split image screen are integrated into one concave surface having a curvature the same as that of a human eye, the examinee can clearly recognize the fixation points at the same time the split image focusing is completed.

The present invention has been described with some preferred embodiments thereof and it is understood that many changes and modifications in the described embodiments can be carried out without departing from the scope and the spirit of the invention that is intended to be limited only by the appended claims.

Whet is claimed is:

1. A gaze-fixation aiding and image focusing device, comprising:
an illuminating system having an illuminating optical path for projecting an examination light to illuminate an examinee's fundus;
an imaging system having an imaging optical path for receiving the examinee's fundus image and light reflected from the examinee's eye, and for forming images of the reflected light and the examinee's fundus on a display;
a focusing device including a split image screen located in the illuminating optical path and an adjusting means located in the imaging optical path to work with the split image screen to enable split image focusing; the split image screen including a light blocking plate of a predetermined size having a light-transmissible slit provided thereon, and two facing prisms being arranged in front of the slit; and
a gaze fixation device including a gaze fixation surface formed in the illuminating optical path for the examinee to gaze on, and a plurality of fixation points provided on the gaze fixation surface with a contrast being created between the examination light and the fixation points;
wherein the split image screen and the fixation points together form a light-impenetrable zone that blocks the illuminating optical path, and areas of the gaze fixation surface other than the light-impenetrable zone together form a light-penetrable zone.

2. The gaze-fixation aiding and image focusing device as claimed in claim 1, wherein the examination light is an invisible light, and the fixation points are respectively provided with at least one light-emitting unit that can be selectively controlled to light or extinguish.

3. The gaze-fixation aiding and image focusing device as claimed in claim 2, wherein the light-emitting units are selected from the group consisting of light-emitting diodes, lamp tubes, light bulbs, and optical fibers.

4. The gaze-fixation aiding and image focusing device as claimed in claim 2, wherein the light-emitting units are in the form selected from the group consisting of geometrical shapes, characters, figures, letters, symbols, patterns, and any combination thereof.

5. The gaze-fixation aiding and image focusing device as claimed in claim 2, wherein the fixation points are connected to one another via a linking unit.

6. The gaze-fixation aiding and image focusing device as claimed in claim 5, wherein the linking unit is a cross-shaped plate extended from the light blocking plate to each of the fixation points.

7. The gaze-fixation aiding and image focusing device as claimed in claim 5, wherein the linking unit includes a horizontal plate, which is integrally outward extended from two opposite lateral sides of the light blocking plate to link with the fixation points; a ring-shaped plate, which is integrally outward extended from two opposite ends of the horizontal plate; and two vertical plates, which are respectively radially inward extended from an upper and a lower end of the ring-shaped plate to link with the fixation points.

8. The gaze-fixation aiding and image focusing device as claimed in claim 5, wherein the linking unit includes a vertical plate, which is integrally radially outward extended from a lower side of the light blocking plate to link with the fixation points; a ring-shaped plate, which is integrally extended from a lower end of the vertical plate to form a circle around the light blocking plate; and three projected plates, which are integrally radially inward extended from an upper, a left and a right end of the ring-shaped plate to link with the fixation points.

9. The gaze-fixation aiding and image focusing device as claimed in claim 1, wherein the adjusting means is configured as a movable focusing lens for selectively adjusting a focal length position.

10. The gaze-fixation aiding and image focusing device as claimed in claim 1, wherein the fixation points and the split image screen are both located on the gaze fixation surface, the gaze fixation surface is configured as a flat vertical surface, and the fixation points are arranged on an upper, a lower, a left and a right side of the vertical gaze fixation surface to locate around and space from the split image screen by a predetermined distance.

11. The gaze-fixation aiding and image focusing device as claimed in claim 10, wherein two more fixation points are arranged on the vertical gaze fixation surface to locate at two opposite lateral sides of the slit on the light blocking plate of the split image screen.

12. The gaze-fixation aiding and image focusing device as claimed in claim 1, wherein the fixation points and the split image screen are both located on the gaze fixation surface, the gaze fixation surface is configured as a concave surface having a curvature corresponding to that of a human eye, and the fixation points are arranged on an upper front, a lower front, a front left and a front right side of the concave surface to locate around the split image screen.

13. The gaze-fixation aiding and image focusing device as claimed in claim 12, wherein two more fixation points are arranged on the front left and the front right side of the concave surface to locate at two opposite lateral sides of the slit of the split image screen.

14. The gaze-fixation aiding and image focusing device as claimed in claim 1, wherein the examination light is a visible light, and the fixation points are respectively provided with at least one lightproof unit.

15. The gaze-fixation aiding and image focusing device as claimed in claim 14, wherein the light-penetrable zone is configured as a transparent substrate, the split image screen being arranged at a center of the transparent substrate, and the lightproof units being arranged on the transparent substrate to locate around and space from the split image screen by a predetermined distance.
